(19) United States
(12) Patent Application Publication
(10) Pub. No.: US 2007/0003589 A1
(43) Pub. Date: Jan. 4, 2007

(54) Coatings for implantable medical devices containing attractants for endothelial cells

(76) Inventors: Irina Astafieva, Palo Alto, CA (US); Thierry Glauser, Redwood City, CA (US); Syed Faiyaz Ahmed Hossainy, Fremont, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/355,870

(22) Filed: Feb. 15, 2006

Related U.S. Application Data
(60) Provisional application No. 60/654,212, filed on Feb. 17, 2005.

(51) Int. Cl. A61K 48/00; A61K 31/4745; A61F 2/02

(52) U.S. Cl. 424/423; 514/44; 514/291

(57) ABSTRACT

Provided herein is a coating that includes a chemo-attractant for endothelial cells and methods of making and using the same.

### CROSS-REFERENCE TO RELATED APPLICATION

This application claims priority to U.S. provisional application No. 60/654,212, filed on Feb. 17, 2005, the teachings of which are incorporated herein by reference.

### BACKGROUND

1. Field of the Invention

This invention is generally related to coatings for implantable medical devices, such as drug delivery vascular stents.

2. Description of the State of the Art

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, ISR still poses a significant problem given the large volume of coronary interventions and their expanding use. The pathophysiological mechanism of ISR involves interactions between the cellular and acellular elements of the vessel wall and the blood. Damage to the endothelium during PCI constitutes a major factor for the development of ISR (see, e.g., Kipshidze, N., et al., J. Am. Coll. Cardiol. 44:733-739 (2004)).

The embodiments of the present invention address these concerns as well as others that are apparent to one having ordinary skill in the art.

### SUMMARY

Provided herein is a coating that includes a chemo-attractant for endothelial cells. The coating is capable of providing controlled release of the chemo-attractant. In some embodiments, the coating can include one or more polymers that are capable of controlling the chemo-attractant's release. The chemo-attractant can diffuse through the polymer coating, through a layer of absorbed proteins and cells (acute phase after implantation), and through the neo-intima (long-term phase) to the lumen surface in an amount sufficient to recruit endothelial cells or endothelial progenitor cells to the surface.

In some embodiments, the release profile includes an initial burst release followed by sustained release of the chemo-attractant. In some other embodiments, the release profile can be a zero-order sustained release.

The chemo-attractant and optionally other bioactive agent(s) can be blended in the coating in the form of a matrix. The chemo-attractant can be formulated into the coating with one or more biocompatible polymer, which, in some embodiments, can be an amphiphilic block copolymer. In some embodiments, the chemo-attractant can be attached to the coating via a physical or chemical linkage. Physical linkages can be, e.g., hydrogen bonding or interpenetrating networks. Chemical linkages can occur through a linking agent.

Any bioactive agent can be included in a coating with the chemo-attractant described herein. Some examples of the bioactive agent include siRNA and/or other oligoneucleotides that inhibit endothelial cell migration. The bioactive agent can also be lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction. Some other exemplary bioactive agents are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, prodrugs thereof, co-drugs thereof, and combinations thereof.

The coating can be formed on an implantable device such as a stent, which can be implanted in a patient to treat, prevent, mitigate, or reduce a vascular medical condition, or to provide a pro-healing effect. Examples of these conditions include atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, or combinations of these.

### DETAILED DESCRIPTION

Provided herein is a coating that includes a chemo-attractant for endothelial cells. The coating is capable of providing controlled release of the chemo-attractant. In some embodiments, the coating can include one or more polymers that are capable of controlling the chemo-attractant's release. The chemo-attractant can diffuse through the polymer coating, through a layer of absorbed proteins and cells (acute phase after implantation), and through the neo-intima (long-term phase) to the lumen surface in an amount sufficient to recruit endothelial cells or endothelial progenitor cells to the surface.

In some embodiments, the release profile includes an initial burst release followed by sustained release of the chemo-attractant. In some other embodiments, the release profile can be a zero-order sustained release.

The chemo-attractant and optionally other bioactive agent(s) can be blended into the coating. In some embodiments, the blend or mixture of chemo-attractant and coating material can form a matrix. The chemo-attractant can be formulated into the coating with one or more biocompatible polymer, which, in some embodiments, can be an amphiphilic block copolymer. In some embodiments, the chemo-attractant can be attached to the coating via a physical or chemical linkage. Physical linkages can be, e.g., hydrogen bonding or interpenetrating networks. Chemical linkages can occur through a linking agent.

In these or other embodiments, the chemo-attractant can be attached to the surface of a medical device. In some of these embodiments, the surface can be a modified metallic surface (e.g., modified with siloxanes) or a polymeric surface if the medical device is formed of a polymer or is formed of a metal coated with a polymer. The chemo-attractant can attach to the coating using a physical or chemical linkage.

Physical linkages can be, e.g., hydrogen bonding, interpenetrating molecules or interpenetrating networks. Chemical linkages can use a linking agent or a direct bond between the surface and the coating material.

Any bioactive agent can be included in a coating with the chemo-attractant described herein. Some examples of the bioactive agent include siRNA and/or other oligoneucleotides that inhibit endothelial cell migration. The bioactive agent can also be lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction. Some other exemplary bioactive agents are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, prodrugs thereof, co-drugs thereof, and combinations thereof.

The coating can be formed on an implantable device such as a stent, which can be implanted in a patient to treat, prevent, mitigate, or reduce a vascular medical condition, or to provide a pro-healing effect. Examples of these conditions include atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, or combinations of these.

### Chemo-Attractants

As used herein, the chemo-attractant includes any synthetic or natural molecules capable of attracting endothelial cells. In some embodiments, the chemo-attractant includes any synthetic or natural molecules capable of attracting an effective number of endothelial cells. The attractant generally has a degree of selectivity towards these cells. The chemo-attractant also includes any synthetic or natural molecules capable of binding to adhesion receptors differentially expressed on the endothelial cells. One such adhesion receptor can be integrin. Some exemplary chemo-attractants include, but are not limited to, small integrin-binding molecules, RGD peptide or cyclic RGD peptide (cRGD), synthetic cyclic RGD (cRGD) mimetics, and small molecules binding to other adhesion receptors differentially expressed on the endothelial cells.

In some embodiments, the chemo-attractant can specifically exclude a particular RGD peptide or cyclic RGD peptide (cRGD).

In some embodiments, the chemo-attractant can be those molecules capable of binding to ICAM (intercellular adhesion molecule) molecules or VCAM (vascular cell adhesion molecule) molecules, which are present in the endothelial cells. Such chemo-attractant can be, for example, receptors binding to ICAM or VCAM in the endothelial cells, which can include, but are not limited to, Decoy receptor 3 (DcR3), a tumor necrosis factor (TNF) that preferentially binds to ICAM and VCAM, β_{—}2 integrin LFA-1. (LFA-1Af) (expressed on lymphocytes), which has conformational changes in extracellular domains enabling higher affinity binding to the ligand ICAM-1, or combinations thereof.

In some embodiments, the chemo-attractant can be used in an encapsulated form, e.g., encapsulation in liposome or another material such as a biodegradable polymer. The encapsulated chemo-attractant can be used in connection with a catheter and, in some embodiments, subsequently be released from the catheter.

### cRGD or RGD Mimetics

The cRGD or RGD mimetics described herein includes any peptides or peptide mimetics that result from the modification of the cyclic Arg-Gly-Asp peptide. The modification can be on the pendant groups and/or on the backbone of the peptide. Peptide synthesis, including the synthesis of peptide mimetics, is well documented and can be readily achieved using, for example, combinatorial chemistry.

Some examples of cRGD or RGD mimetics include αᵥβ₃ antagonist such as IIb/IIIb antagonists (Coller, B. S., Thromb. Haemost. 86(1):427-43 (2001) (Review)), one example of which is Abciximax (Blindt, R., J. Mol. Cell. Cardiol. 32:2195-2206 (2000)), XJ 735 (Srivatsa, S. S., et al., Cardiovasc. Res. 36:408-428 (1997)), anti-β₃-integrin antibody F11, cRGD (Sajid, M., et al., Am. J. Physiol. Cell Physiol., 285:C1330-1338 (2003), and other sequences such as laminin derived SIKVAV (Fittkau, M. H., et al., Biomaterials, 26:167-174 (2005)), laminin derived YIGSR (Kouvroukoglou, S., et al., Biomaterials, 21:1725-1733 (2000)), KQAGDV, and VAPG (Mann, B. K., J. Biomed. Mater. Res. 60(1):86-93 (2002))

The following describes a basic procedure for the synthesis of a peptide, including a peptide mimetics:

Before the peptide synthesis starts, the amine end of the amino acid (starting material) is protected with FMOC (9-fluoromethyl carbamate) or other protective groups, and a solid support such as a Merrifield resin (free amines) is used as an initiator. Then, step (1) through step (3) reactions are performed and repeated until the desired peptide is obtained: (1) a free amine is reacted with the carboxyl end using carbodiimide chemistry, (2) the amino acid sequence is purified, and (3) the protecting group, e.g., the FMOC protecting group, is removed under mildly acidic conditions to yield a free amine. The peptide can then be cleaved from the resin to yield a free standing peptide or peptide mimetic.

In some embodiments, a coating can specifically exclude any of the above mentioned chemo-attractant. For example, a coating can specifically exclude RGD peptide or cyclic RGD peptide (cRGD).

### Linkers

In some embodiments, the chemo-attractant can be attached to a polymer matrix via a labile linker or via physical interactions such as interpenetrating networks. The labile linker can be a linker sensitive to a stimulus. For example, the linker can be a hydrolytically degradable linker or an enzymetically degradable linker.

Hydrolytically degradable linkers can degrade under physiological conditions in the presence of water. In other words, the stimulus for a hydrolytically degradable linker is the presence of water. A hydrolytically degradable linker can link the chemo-attractant and the polymer via the linker's reactive groups. For example, in some embodiments, the linker can be an amino acid group that includes amino, thiol, and/or carboxylic groups. Some exemplary strategies for forming hydrolytically degradable linkers include:

(1) ε-Amino group of lysine (which can be integrated into a polymer) and α-amino group of a protein. The amine can be on the polymer backbone (with or without a spacer, e.g., PEG, or an alkyl chain). This can yield an amide, thiourea, alkylamine or urethane linkage.

(2) Thiol group or a free cysteine, which forms a thioether linkage.

(3) Thiol group on a cysteine, which can be conjugated with vinylsulfone (R—SO₂—CH═CH₂).

(4) Carboxylic acid groups on the aspartic and glutamic acid.

Some examples of hydrolytically degradable linkages include amide linkages that can be generated by reacting an amine group with succinate esters such as N-hydroxysuccinimide (NHS), thiol linkages such as disulfide (R-L1-S-S-L2-R′) where the length of the linker L1 and L2 control the hydrolization, or ester bonds formed by coupling the peptide's carboxylic end with a hydroxyl on the polymer backbone (with or without a spacer, e.g., PEG, or an alkyl chain). Esterification can be carried out using established methods in the art (e.g., carbodiimide chemistry in the presence of 1-ethyl-3-(3-dimethylaminopropyl)carbodiimide (EDC)).

Enzymatically degradable linkers/linkages can be degraded by an enzyme, often to target a specific area of the body or organ. In other words, the stimulus for an enzymatically degradable linker is the presence of an enzyme. For example, a specific dipeptide sequence can be incorporated into the linker, which can be cleaved by an enzyme. Some examples of enzymetically degradable linkers or linkages include, but are not limited to, self-immolative p-aminobenzyloxycarbonyl (PABC) spacer between the dipeptide and the polymer, dipeptides such as phenylaniline-lysine and valine-cysteine, or PEG/dipeptide linkages such as alanyl-valine, alanyl-proline and glycyl-proline.

Some other linker/linkages can be found at “Biodegradable Polymers for Protein and Peptide Drug Delivery” Bioconjugate Chem. 1995, 6:332-351; M. P. Lutolf and J. A. Hubbell, Biomacromolecules 2003, 4:713-722; and U.S. patent application Ser. No. 10/871,658. Some additional representative linking chemistry is described in U.S. patent application Ser. No. 10/871,658, the entire disclosure of which is hereby incorporated by reference.

### Biocompatible Polymers

Any biocompatible polymer can form a coating with a chemo-attractant described herein. The biocompatible polymer can be biodegradable (both bioerodable or bioabsorbable) or nondegradable and can be hydrophilic or hydrophobic.

Representative biocompatible polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide/poly(lactic acid) (PEO/PLA)), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as chitosan, alginate, fibrin, fibrinogen, cellulose, starch, dextran, dextrin, fragments and derivatives of hyaluronic acid, heparin, fragments and derivatives of heparin, glycosamino glycan (GAG), GAG derivatives, polysaccharide, chitosan, alginate, or combinations thereof. In some embodiments, the copolymer described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Biobeneficial Material

In some embodiments, the chemo-attractant can be included in a coating with a biobeneficial material. The combination can be mixed, blended, or patterned or arranged in separate layers. The biobeneficial material useful in the coatings described herein can be polymeric or non-polymeric. The biobeneficial material is preferably non-toxic, non-antigenic and non-immunogenic enough so that it can be successfully introduced into a patient. A biobeneficial material is one which enhances the biocompatibility of a device by being non-fouling, hemocompatible, actively non-thrombogenic, or anti-inflammatory, all without depending on the release of a pharmaceutically active agent.

Representative biobeneficial materials include, but are not limited to, polyethers such as poly(ethylene glycol), copoly(ether-esters), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly (ethylene glycol) acrylate (PEGA), PEG methacrylate, 2-methacryloyloxyethylphosphorylcholine (MPC) and n-vinyl pyrrolidone (VP), carboxylic-acid-bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, chitosan, alginate, silicones, PolyActive™, and combinations thereof. In some embodiments, the coating can exclude any one of the aforementioned polymers.

The term PolyActive™ refers to a block copolymer having flexible poly(ethylene glycol) and poly(butylene terephthalate) blocks (PEGT/PBT). PolyActive™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

In a preferred embodiment, the biobeneficial material can be a polyether such as poly (ethylene glycol) (PEG) or polyalkylene oxide.

### Bioactive Agents

In some embodiments, a coating having one or more chemo-attractants described herein can optionally include one or more bioactive agents. These bioactive agents can be any agent which is a therapeutic, prophylactic, or diagnostic agent. These agents can have anti-proliferative or anti-inflammmatory properties or can have other properties such as antineoplastic, antiplatelet, anti-coagulant, anti-fibrin, antithrombonic, antimitotic, antibiotic, antiallergic, or antioxidant properties.

These agents can be cystostatic agents, agents that promote the healing of the endothelium (other than by releasing or generating NO), or agents that promote the attachment, migration and proliferation of endothelial cells while quenching smooth muscle cell proliferation. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules, which bind to complementary DNA to inhibit transcription, and ribozymes. Some other examples of biobactive agents include antibodies, receptor ligands, enzymes, adhesion peptides, blood clotting factors, inhibitors or clot dissolving agents, such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include ABT-578, 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include biolimus, tacrolimus, dexamethasone, clobetasol, corticosteroids or combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, pimecrolimus, imatinib mesylate, midostaurin, and genetically engineered epithelial cells. The foregoing substances can also be used in the form of prodrugs or co-drugs thereof. The foregoing substances also include metabolites thereof and/or prodrugs of the metabolites. The foregoing substances are listed by way of example and are not meant to be limiting. Other active agents which are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutically effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by those of ordinary skill in the art.

### Examples of Implantable Device

As used herein, an implantable device can be any suitable medical substrate that can be implanted in a human or veterinary patient. Examples of such implantable devices include self-expandable stents, balloon-expandable stents, stent-grafts, grafts (e.g., aortic grafts), heart valve prosthesis (e.g., artificial heart valves) or vascular graft, cerebrospinal fluid shunts, pacemaker electrodes, catheters, endocardial leads (e.g., FINELINE and ENDOTAK, available from Guidant Corporation, Santa Clara, Calif.), and devices facilitating anastomosis such as anastomotic connectors. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE-(Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. The device can be, for example, a bioabsorbable stent.

### Method of Use

In accordance with embodiments of the invention, a chemo-attractant can be included in an implantable device or prosthesis, e.g., a stent. For a device including one or more active agents, the agent will retain on the device such as a stent during delivery and expansion of the device, and released at a desired rate and for a predetermined duration of time at the site of implantation.

Preferably, the device is a stent. The stent described herein is useful for a variety of medical procedures, including, by way of example, treatment of obstructions caused by tumors in the bile ducts, esophagus, trachea/bronchi and other biological passageways. A stent having the above-described coating is particularly useful for treating occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter that allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A medical device comprising a chemo-attractant for endothelial cells.

2. The medical device of claim 1 wherein the chemo-attractant is not an RGD or cyclic RGD peptide.

3. The medical device of claim 1 having a coating comprising a polymer and the chemo-attractant.

4. The medical device of claim 1 wherein the chemo-attractant binds to an adhesion receptor differentially expressed on the endothelial cells.

5. The medical device of claim 1 wherein the chemo-attractant is a receptor that binds to an intercellular adhesion molecule (ICAM) or a vascular cell adhesion molecule (VCAM).

6. The medical device of claim 1 wherein the chemo-attractant is Decoy receptor 3 (DcR3), β_{—}2 integrin LFA-1 (LFA-1Af), or a combination thereof.

7. The medical device of claim 4 wherein the adhesion receptor is integrin.

8. The medical device of claim 3 wherein the chemo-attractant binds to an adhesion receptor differentially expressed on the endothelial cells.

9. The medical device of claim 8 wherein the adhesion receptor is integrin.

10. The medical device of claim 1 wherein the chemo-attractant is a cRGD or RGD mimetic.

11. The medical device of claim 8 wherein the chemo-attractant is a cRGD or RGD mimetic.

12. The medical device of claim 3 wherein a linker attaches the chemo-attractant to the polymer.

13. The medical device of claim 12 wherein the linker is a hydrolytically degradable linker or a proteolytically degradable linker.

14. The medical device of claim 12 wherein the linker is an enzymetically degradable linker.

15. The medical device of claim 12 wherein the linker comprises poly(ethylene glycol) (PEG) or an alkyl chain.

16. The medical device of claim 13 wherein the hydrolytically degradable linker is selected from the group consisting of an amide linkage, a thiol linkage, an ester linkage, a thiourea linkage, an alkylamine linkage, a urethane linkage, a thioether linkage and combinations thereof.

17. The medical device of claim 13 wherein the hydrolytically degradable linker comprises a cysteine unit, an aspartate unit, a glutamate unit, or combination thereof.

18. The medical device of claim 12 wherein the linker is a biodegradable polymer.

19. The medical device of claim 14 wherein the enzymetically degradable linker comprises a dipeptide sequence.

20. The medical device of 14 wherein the enzymetically degradable linker comprises a spacer.

21. The medical device of claim 20 wherein the spacer is selected from the group consisting of p-aminobenzyloxycarbonyl (PABC), a dipeptide, PEG, and combinations thereof, and
wherein the dipeptide is selected from the group consisting of phenylaniline-lysine, valine-cysteine, alanyl-valine, alanyl-proline, glycyl-proline and combinations thereof.

22. The medical device of claim 12 wherein the linker is a physical linker.

23. The medical device of claim 1 wherein the chemo-attractant is encapsulated in a liposome or a biodegradable polymer.

24. The medical device of claim 23 wherein the chemo-attractant is capable of release from a catheter.

25. The medical device of claim 1 wherein the chemo-attractant has a release profile that includes an initial burst release followed by sustained release.

26. The medical device of claim 1 wherein the chemo-attractant has a release profile which is zero-order sustained release.

27. The medical device of claim 3, further comprising a bioactive agent.

28. The medical device of claim 3, further comprising a bioactive agent selected from the group consisting of paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, midostaurin, prodrugs thereof, co-drugs thereof, and combinations thereof.

29. The medical device of claim 3, further comprising a bioactive agent selected from the group consisting of siRNA, oligonucleotides that inhibit migration of endothelial cells, lysophosphatidic acid (LPA), sphingosine-1-phosphate (S1P), prodrugs thereof, co-drugs thereof, and combinations thereof.

30. The medical device of claim 1 which is a stent.

31. The medical device of claim 3 which is a stent.

32. The medical device of claim 28 which is a stent.

33. The medical device of claim 1 which is a bioabsorbable stent.

34. The medical device of claim 28 which is a bioabsorbable stent.

35. A method comprising implanting the medical device of claim 1.
